(19) United States
(12) Patent Application Publication
(10) Pub. No.: US 2010/0191043 A1
(43) Pub. Date: Jul. 29, 2010

(54) METHODS AND DEVICES FOR ISOLATING A VESSEL

(75) Inventor: Albert K. Chin, Palo Alto, CA (US)

Correspondence Address: Vista IP Law Group, LLP (Maquet), 1885 Lundy Avenue, Suite 108, San Jose, CA, 95131, US

(73) Assignee: MAQUET CARDIOVASCULAR LLC, San Jose, CA (US)

(21) Appl. No.: 12/693,966

(22) Filed: Jan. 26, 2010

Related U.S. Application Data
(60) Provisional application No. 61/147,544, filed on Jan. 27, 2009.

(51) Int. Cl. A61M 29/00

(52) U.S. Cl. 600/36

(57) ABSTRACT

A method of harvesting a target vessel includes creating a first incision, inserting a light source into a vessel through the first incision, the vessel located next to the target vessel, using the light source to illuminate a portion of the vessel, locating the target vessel from outside the vessel using the illuminated portion of the vessel, and dissecting at least a portion of the target vessel from surrounding tissue. A method that involves a target vessel includes inserting a light source into a vessel that is located next to the target vessel, providing light inside the vessel using the light source, and using light transmitted through a wall of the vessel to illuminate the target vessel or tissue next to the target vessel.

### RELATED APPLICATION DATA

This application claims priority to and the benefit of U.S. Provisional Patent Application No. 61/147,544, filed on Jan. 27, 2009, the entire disclosure of which is expressly incorporated by reference herein.

### FIELD

The present application relates to methods and devices for locating, isolating, dissecting, and/or harvesting a vessel, such as an internal mammary artery, using a light emitting device.

### BACKGROUND

The internal mammary arteries, also known as the internal thoracic arteries, are sometimes used as a conduit in coronary artery bypass surgery. During such surgery, at least a portion of the artery is dissected from surrounding tissue, any side branches extending from the artery are ligated and transected, the artery is severed at least at the distal end, and the severed end is connected to the diseased coronary artery via anastomosis to provide the desired bypass. U.S. Pat. No. 5,797,946, which is incorporated herein by reference, describes a surgical technique of isolating and dissecting an internal mammary artery by making a subcostal incision to expose the superior epigastric artery, which is a continuation of the internal mammary artery. A dissection cannula such as the VasoView vessel harvesting system (MAQUET Cardiovascular LLC, San Jose, Calif.) is then introduced through the subcostal incision and advanced along the epigastric artery and internal mammary artery in the superior direction to form a working cavity along the length of the internal mammary artery, substantially from the inferior epigastric artery to its origin from the subclavian artery. Within the working cavity via the subcostal incision, the arterial side branches as well as the venous tributaries of the adjacent internal mammary vein may be ligated and transected to prepare the internal mammary artery pedicle for anastomosis of the severed end of the internal mammary artery to a coronary artery via a separate thoracotomy incision. The pedicle includes the internal mammary artery, its accompanying vein and connective tissue around the artery and vein. Alternatively, the internal mammary artery may be dissected alone and its free end anastomosed to a coronary artery.

For obese patients, a subcostal approach to internal mammary artery dissection may be difficult because the rigid, straight dissecting endoscope of commercially available vessel harvesting systems may be unable to lie axially against the internal mammary arteries. While a supraclavicular approach to internal mammary dissection would avoid this possible problem, it can be a difficult approach because the internal mammary artery is not easily located through a supraclavicular incision. Therefore, applicant determines that there is a need for an improved surgical technique in which the internal mammary arteries are located and dissected through a superior incision.

### SUMMARY

In one aspect, a method of locating an internal mammary vein within a body is provided. Embodiments of the method include the acts of making a subcostal incision, locating the epigastric vein through the subcostal incision, inserting a light emitting device into an epigastric vein, advancing the light emitting device within the epigastric vein in a superior direction such that the device passes into an internal mammary vein, making a supracostal incision, and using the light emitted by the light emitting device to locate the internal mammary vein through the supracostal incision.

In another aspect, a method of performing bypass surgery on a diseased coronary artery is provided. Embodiments of the method include the steps of making a subcostal incision, locating the epigastric vein through the subcostal incision, inserting a light emitting device into an epigastric vein, advancing the light emitting device within the epigastric vein in a superior direction such that the device passes into an internal mammary vein, making a supracostal incision, using the light emitted by the light emitting device to locate the internal mammary vein through the supracostal incision, dissecting at least a portion of the internal mammary vein from surrounding tissue such that at least a portion of an internal mammary artery is dissected from surrounding tissue, and connecting the internal mammary artery to the diseased coronary artery.

In accordance with some embodiments, a method of harvesting a target vessel includes creating a first incision, inserting a light source into a vessel through the first incision, the vessel being located next to the target vessel, using the light source to illuminate a portion of the vessel, locating the target vessel from outside the vessel using the illuminated portion of the vessel, and dissecting at least a portion of the target vessel from surrounding tissue.

In accordance with other embodiments, a method that involves a target vessel includes inserting a light source into a vessel that is located next to the target vessel, providing light inside the vessel using the light source, and using light transmitted through a wall of the vessel to illuminate the target vessel or tissue next to the target vessel.

Other and further aspects and features will be evident from reading the following detailed description of the embodiments, which are intended to illustrate, not limit, the invention.

### BRIEF DESCRIPTION OF THE DRAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments and are not therefore to be considered limiting of its scope.

FIG. 1A illustrates a method for locating and/or using the internal mammary vessels in accordance with some embodiments.

FIG. 1B is a partial anatomical drawing illustrating the course of the internal mammary artery and an insertion of a light emitting device through a sub-costal incision.

FIG. 2 shows a first embodiment of a light emitting device for use with embodiments of the methods described herein.

FIG. 3 shows a second embodiment of a light emitting device for use with embodiments of the methods described herein.

FIG. 4 shows a dissector device for use with embodiments of the methods described herein.

### DETAILED DESCRIPTION OF THE EMBODIMENTS

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated.

Embodiments of the methods and devices described herein allow for the surgical location of a vessel, such as an internal mammary artery (IMA), and subsequent surgical procedures thereon through an incision, such as a supracostal incision. In accordance with some embodiments, a light emitting device is used to facilitate the visualization of an IMA, or the vessels adjacent an IMA, in a closed-chest surgical procedure. Such visualization allows for the isolation, dissection, and/or harvesting of an IMA more easily than existing techniques. The technique described herein is particularly useful in obese patients, or other patients for whom accessing an IMA from a subcostal incision is difficult.

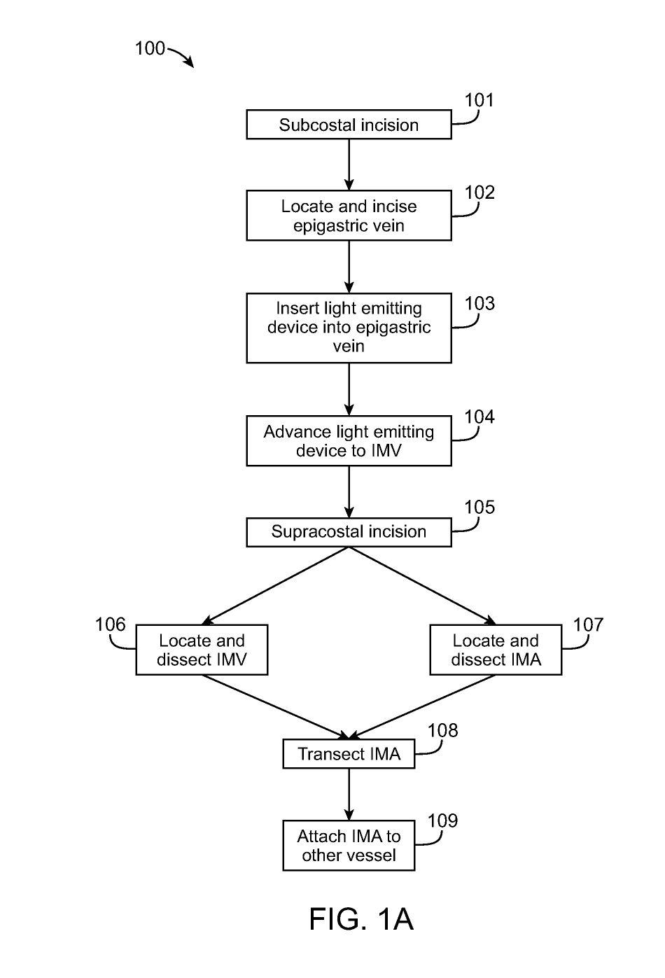

Methods for locating an internal mammary vein (IMV) or IMA in accordance with some embodiments are shown in FIGS. 1A and 1B. In some embodiments of the method 100, a subcostal incision 150 is made (step 101) to locate the superior epigastric vein. In some embodiments, locating the superior epigastric vein is preferred because of its proximity to the IMAs. In other embodiments, the inferior epigastric vein may be located instead. The preferred length of the incision is approximately 2 cm, and the epigastric vein is located through such incision 150 using known surgical techniques (step 102), which may include identifying the epigastric artery with the assistance of a handheld Doppler ultrasound device. The epigastric vein lies adjacent to the epigastric artery, so identification of the artery leads to easy location of the vein.

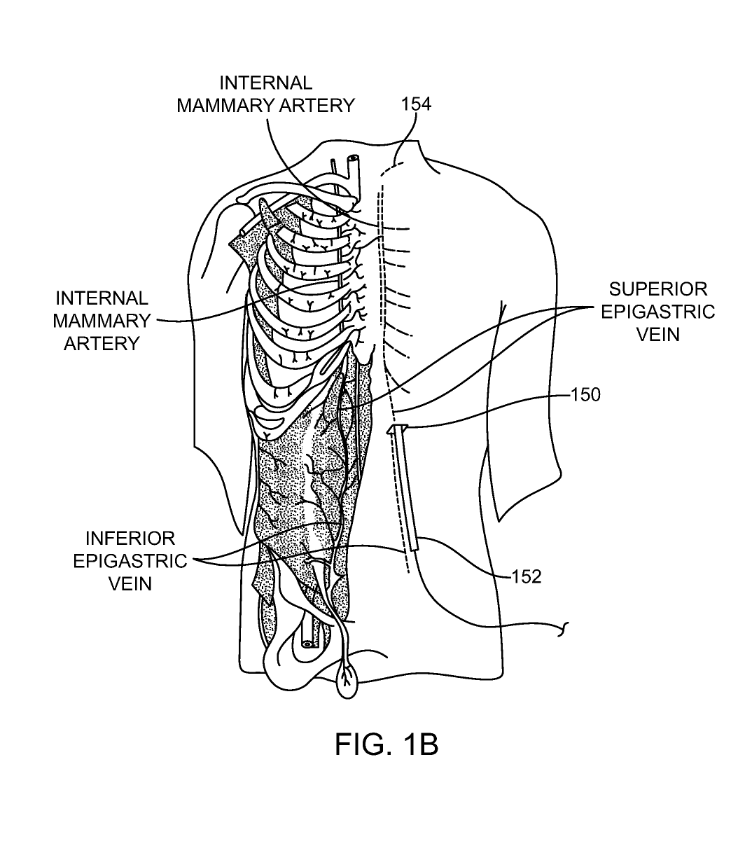

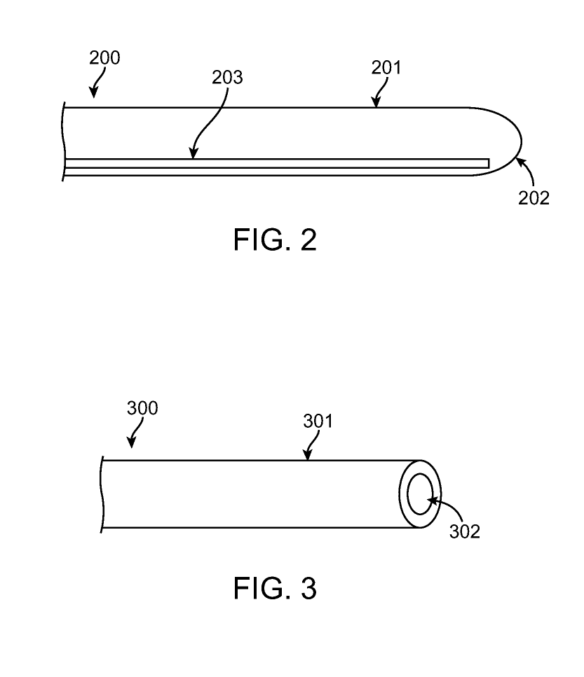

As shown in FIGS. 1A and 1B, once the epigastric vein is located, it is incised and a light emitting device 152 is inserted into the vein (step 103). The light emitting device 152 is preferably a light emitting catheter such as the device described in U.S. Pat. Nos. 7,396,354, 7,366,563, 7,211,040, 6,685,666, 6,236,879, and 5,728,092, which are incorporated herein by reference. An example of a light emitting device 152 that may be used with embodiments of the methods described herein is shown in FIG. 2, which illustrates a light emitting device 200 that comprises a flexible catheter 201 having a closed end 202 and a light source such as a fiber optic cable 203 extending along the length thereof. Alternatively, the light emitting device 152 is a flexible endoscope 300 comprising an elongated cannula 301 having a light 302 at the distal end thereof, as is known in the art, or other similar device. The light emitting device should emit light at or near its distal end, and preferably, emits light along at least some of the length thereof. The light emitting device may be a catheter, such as shown in FIG. 2, containing fiber optic strands that are connected to a light source; for example, a light source with a xenon bulb. The light may also be colored green, yellow, etc. to render it more visible as it is transmitted through the vein wall. The light emitted from the light emitting device is preferably strong enough to be visible through the vein wall.

Once inserted, the light emitting device 152 is advanced in a superior direction, or antegrade to venous blood flow, into the IMV (step 104) and preferably, to the subclavian or internal jugular vein. The IMV runs immediately adjacent to the IMA. The light emitting device 152 is passed within the IMV rather than the IMA because the passage of any object through a blood vessel can damage the blood vessel, for example, by inadvertent puncture or by damaging the endothelial lining or other cells within the internal lumen of the vessel, and the IMA should be preserved for subsequent surgical procedures such as coronary bypass surgery. In this way, the IMV is sacrificed to provide for the use of the IMA in subsequent surgical procedures.

As shown in FIGS. 1A and 1B, following the placement of the light emitting device 152 within the IMV or internal jugular vein, a superior, or supracostal, incision 154 is made (step 105), preferably at the neck or the supraclavicular area. The preferred length of the incision 154 is approximately 2 cm, and the transillumination of the light emitted from the light emitting device 152 through the wall of the IMV is visualized. Visualization of the illuminated IMV from outside the IMV may occur directly using known surgical techniques in the event that the light emitting device 152 is advanced to the internal jugular vein, or endoscopically if advanced to the IMV. In other embodiments, instead of creating the incision 154, the same incision 150 may be used to provide access for viewing the illuminated IMV from outside the IMV.

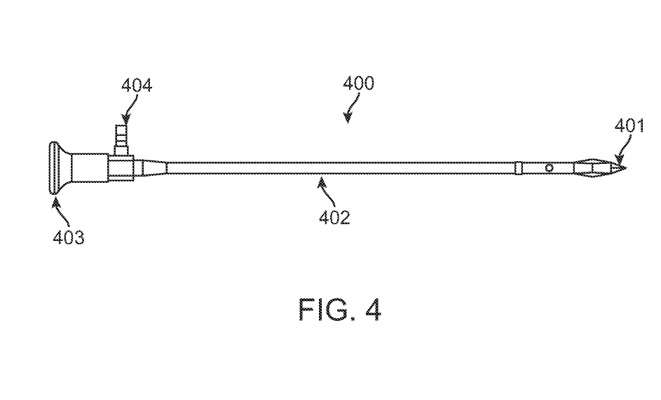

In some embodiments, the IMV is thereafter dissected (step 106) from surrounding tissue through the supracostal incision 154 (or the incision 150) using devices and procedures known in the art. Preferably, the IMV is dissected using the conical blunt tip of the VasoView vessel harvesting system (Maquet Cardiovascular LLC, San Jose, Calif.) in an endoscopic procedure, an embodiment of which is illustrated in FIG. 4. Such devices and procedures are described in U.S. Pat. No. 5,873,889, which is incorporated herein by reference. In this embodiment, the conical tip 401 of dissector 400 is advanced in an inferior direction along a length of the IMV to separate the vessel from adjacent tissue. The dissector 400 includes an elongated cannula 402, an eyepiece 403 at the proximal end thereof, and an illumination port 404. In one embodiment where the light emitting device 152 emits light along the length thereof, the light transmitted through the IMV wall may be used as a guide for the dissection procedure such that the operator follows the path established by the light, as well as tracking along the IMV visually, as in standard surgical practice with the endoscopic vessel harvesting system. Since the IMA is located next to the IMV, tracking the illuminated IMV would lead to the tracking of the IMA. Alternatively, where the light emitting device 152 emits light from only the distal end thereof, it may be withdrawn from the subcostal incision as dissection occurs through the supracostal incision, such that the operator follows the light transmitted through the IMV wall as it advances in an inferior direction.

Because of the proximity of the IMA to the IMV, dissection of the IMV (step 106) results in dissection of the IMA from surrounding tissue. In another embodiment, the IMA is visualized and directly dissected (step 107) as a result of illumination of the IMA from light transmitted through the IMV wall. In either case, the result is that the IMA is dissected from surrounding tissue. The IMV need not be separated from the IMA.

In some embodiments, any side branches extending from the IMA and IMV are transected following the dissection procedure (step 108). Transection of side branches is performed using known endoscopic surgical techniques. To prevent or minimize bleeding from the transected side branches, cauterization is preferably performed before or during transection. In some embodiments, an instrument that includes a jaw assembly, electrodes on the jaw assembly, and a cutter may be inserted into the supracostal incision 154 or the incision 150 to cut and seal the side branches. During use, the jaw assembly is used to clamp against the side branch, and the electrodes are activated to seal the side branch. After the side branch is sealed, the cutter is used to cut the side branch. Alternatively, ligation clips are placed on the side branches prior to transection.

The result of the dissection and side branch transection procedures is an IMA that has at least a portion that is substantially free and separated from adjacent tissues. Alternatively, the IMA remains attached to the IMV. In either case, the dissected IMA is transected to form a distal severed end while leaving the proximal end attached. Transection of the IMA is conducted using devices and procedures known in the art. For example, transection of the IMA can occur endoscopically using endoscopic scissors or other cutting devices. Alternatively, transection of the IMA occurs by making one or more incisions into the thoracic cavity to access the dissected IMA to facilitate the cutting thereof.

In other embodiments, the IMA is severed at both proximal and distal ends, and harvested such that it is removed from the body. Removal occurs by grasping the severed section of the IMA, either endoscopically or directly, through either of the supracostal or subcostal incisions, and pulling from the body. The harvested vessel may thereafter be attached to another vessel (step 109) to be used as a fluid conduit as a free graft elsewhere in the body, such as may be the case in coronary bypass surgery. In other embodiments, the IMA is not removed from the body, but instead is used in an in situ surgical procedure. For example, the IMA is used in an in situ coronary bypass procedure, in which the severed end of the IMA is attached directly to a diseased coronary artery in a closed-chest, endoscopic bypass or MIDCAB (minimally invasive direct coronary artery bypass) procedure using known surgical techniques.

The methods and devices according to the embodiments described herein allow for the reliable and reproducible surgical location of the internal mammary vein and/or artery from supracostal incisions, making them available for subsequent dissection and use in subsequent surgical procedures such as coronary bypass surgery by techniques not previously known.

In other embodiments, the placement of the light emitting device 152 may be reversed. For example, in other embodiments, the light emitting device 152 may be inserted through the superior, or supracostal, incision 154, and the instrument for performing the medical procedure on the target vessel may be inserted through the incision 150. In further embodiments, both the light emitting device 152 and the instrument for performing the medical procedure on the target vessel may be inserted through the incision 150, or through the incision 154. In still further embodiments, the light emitting device and the instrument for performing the medical procedure on the target vessel may be inserted through other incision(s) at other bodily location(s).

In the above embodiments, the method has been described with reference to locating and/or harvesting a vessel. However, in other embodiments, techniques described herein may be used to locate other types of tissue, and/or to perform other types of procedures on tissue. For example, in other embodiments, the light emitting device may be inserted into a vessel to illuminate tissue outside the vessel, thereby allowing a surgeon to identify target tissue next to the vessel. In some cases, additional medical procedures may be performed on the target tissue that is identified by the light illuminated through the vessel wall.

Although particular embodiments have been shown and described, it will be understood that they are not intended to limit the present inventions, and it will be obvious to those skilled in the art that various changes and modifications may be made without departing from the spirit and scope of the present inventions. The specification and drawings are, accordingly, to be regarded in an illustrative rather than restrictive sense. The present inventions are intended to cover alternatives, modifications, and equivalents, which may be included within the spirit and scope of the present inventions as defined by the claims.

1. A method of harvesting a target vessel, comprising:
creating a first incision;
inserting a light source into a vessel through the first incision, the vessel located next to the target vessel;
using the light source to illuminate a portion of the vessel;
locating the target vessel from outside the vessel using the illuminated portion of the vessel; and
dissecting at least a portion of the target vessel from surrounding tissue.

2. The method of claim 1, wherein the act of locating the target vessel comprises using an illumination of the target vessel from light transmitted through a wall of the vessel.

3. The method of claim 1, wherein the vessel comprises an epigastric vein.

4. The method of claim 3, further comprising advancing the light source within the epigastric vein in a superior direction such that the light source passes into an internal mammary vein.

5. The method of claim 1, wherein the target vessel comprises an internal mammary artery.

6. The method Of claim 1, wherein the incision comprises a subcostal incision.

7. The method of claim 1, further comprising making a second incision, wherein the act of dissecting the at least a portion of the target vessel comprises inserting an instrument into the second incision.

8. The method of claim 7, wherein the instrument comprises an endoscopic cutting device.

9. The method of claim 7, wherein the second incision comprises a supracostal incision.

10. The method of claim 1, wherein the at least a portion of the target vessel that is dissected is attached to the vessel.

11. The method of claim 1, wherein the act of using the light source comprises providing a green light or a yellow light.

12. The method of claim 1, further comprising cutting a first end of the target vessel.

13. The method of claim 12, further comprising cutting a second end of the target vessel.

14. The method of claim 1, wherein the act of dissecting comprises accessing the target vessel endoscopically.

15. The method of claim 1, wherein the act of dissecting comprises viewing the target vessel directly through the incision or another incision.

16. The method of claim 1, wherein the act of dissecting comprises cutting side branches that are coupled to the target vessel, and sealing the side branches.

17. A method that involves a target vessel, comprising:
inserting a light source into a vessel that is located next to the target vessel;
providing light inside the vessel using the light source; and
using light transmitted through a wall of the vessel to illuminate the target vessel or tissue next to the target vessel.

18. The method of claim 17, wherein the vessel comprises an epigastric vein.

19. The method of claim 18, further comprising advancing the light source within the epigastric vein in a superior direction such that the light source passes into an internal mammary vein.

20. The method of claim 17, wherein the target vessel comprises an internal mammary artery.

21. The method of claim 17, further comprising making an incision, wherein the light source is inserted using the incision.

22. The method of claim 21, wherein the incision comprises a subcostal incision.

23. The method of claim 17, wherein the light comprises a green light or a yellow light.

24. The method of claim 17, further comprising performing a procedure on the target vessel.

25. The method of claim 24, wherein the procedure involves dissecting at least a portion of the target vessel from surrounding tissue.

26. The method of claim 25, wherein the at least a portion of the target vessel that is dissected is attached to the vessel.

27. The method of claim 25, wherein the act of dissecting comprises cutting side branches that are coupled to the target vessel, and sealing the side branches.

28. The method of claim 24, wherein the procedure comprises:
making an incision; and
inserting an instrument into the incision.

29. The method of claim 28, wherein the instrument comprises an endoscopic cutting device.

30. The method of claim 28, wherein the incision comprises a supracostal incision.

31. The method of claim 24, wherein the procedure comprises cutting a first end of the target vessel.

32. The method of claim 31, further comprising cutting a second end of the target vessel.
